(19) United States
(12) Patent Application Publication
(10) Pub. No.: US 2010/0217268 A1
(43) Pub. Date: Aug. 26, 2010

(54) INTERVERTEBRAL MILLING INSTRUMENT

(75) Inventors: Roy D. Bloebaum, Salt Lake City, UT (US); Sujeevini Jeyapalina, Salt Lake City, UT (US); Amie M. Tanner, Salt Lake City, UT (US); Julian C. Bowman, Midvale, UT (US)

Correspondence Address: KELLY LOWRY & KELLEY, LLP, 6320 CANOGA AVENUE, SUITE 1650, WOODLAND HILLS, CA, 91367, US

(73) Assignees: UNIVERSITY OF UTAH, Salt Lake City, UT (US); U. S. DEPARTMENT OF VETERANS AFFAIRS, Washington, DC (US)

(21) Appl. No.: 12/709,265

(22) Filed: Feb. 19, 2010

Related U.S. Application Data
(60) Provisional application No. 61/208,128, filed on Feb. 20, 2009.

(51) Int. Cl. A61B 17/00

(52) U.S. Cl. 606/83

(57) ABSTRACT

An intervertebral milling instrument for removing calcified fibrous cartilage (CFC) from adjacent vertebrae during spinal fusion or spacer implantation surgery. The milling instrument includes a grinding wheel carried within a guide housing sized to fit closely into the intervertebral space, and to expose the grinding wheel on opposite sides thereof for controlled removal of CFC layers from adjacent cortical end plates. The grinding wheel is rotatably driven as by attachment to a surgical drill, preferably to include saline supply to and suction removal from the grinding site. The controlled removal of CFC layers is limited to about 100 to about 250 microns, thereby avoiding exposing the cortical end plates without excessive bone removal. The instrument is conveniently provided in a kit having multiple guide housings and associated grinding wheels of different sizes to fit into different intervertebral spaces.

### BACKGROUND OF THE INVENTION

This invention relates generally to an improved surgical instrument for controlled milling or shaving to remove calcified fibrous cartilage (CFC) from adjacent vertebrae in the course of spinal surgery such as fusion or spacer implantation surgery. The improved instrument comprises a compact guide housing having a size selected to fit closely into the intervertebral space, wherein the guide housing carries a small grinding wheel that is partially exposed and protrudes a short distance beyond the opposite side faces of the guide housing for engaging and removing thin CFC layers from the cortical endplates of adjacent vertebrae in a closely controlled manner. Excessive bone removal and resultant undesirable exposure of softer cancellous bone is thereby avoided. In a preferred form, the surgical instrument is provided in a kit comprising multiple different guide housings of selected different widths, with the surgeon selecting a specific instrument guide housing having a width suitable for the particular patient application.

Spinal surgery is rapidly becoming a treatment of choice to relieve painful spinal joints and/or to correct joint deformities such as spondylosis (spinal degenerative changes) and disk disorders. In this regard, such spinal surgery typically involves removal of natural cartilage within an intervertebral space, wherein this natural cartilage has degenerated or otherwise broken down to result in patient pain or discomfort, followed by replacement of the natural cartilage with an intervertebral cage or spacer element. Such intervertebral cage or spacer elements may take the form of a rigid component resulting in fixation or fusion of the adjacent vertebral structures, or a mobile bearing disk which is capable of accommodating some relative motion between the adjacent vertebral structures. In either case, it is highly desirable for the spacer element to be securely attached or fixed to the adjacent vertebrae as by direct bone ingrowth to prevent spacer element migration into interfering relation with the patient's spinal cord, or in an opposite direction out of the intervertebral space. Indeed, in one common design, a generally U-shaped rigid spacer element is pre-packed with autologous or autogenous bone cells designed for improved ingrowth attachment with the adjacent vertebral bone structures.

It has been discovered, however, that the adjacent vertebral bone structures are coated with a relatively thin layer of calcified fibrous cartilage (CFC) which is normally beneficial for securely attaching the natural cartilage between the vertebral bones. However, such CFC layer is not conducive to secure osseointegration attachment to a spacer element installed into the intervertebral space following removal of the natural cartilage. That is, the CFC layer exhibits a marked inability to resist bone remodeling or attachment ingrowth thereby resulting in a significant increase in the risk of inadequate spacer element attachment and resultant undesirable post-surgical loosening of the spacer element.

In that past, various so-called bone grinding and/or removal devices have been proposed for preparing bone surfaces for remodeling and/or ingrowth into an adjacent prosthetic device. While such prior art devices may or may not have recognized the need to remove the thin CFC layer from the patient bone in order to achieve this desirable bone ingrowth, the prior art has generally failed to recognize or appreciate that excessive bone removal can also lead to ingrowth complications and failures. That is, prior art bone removal devices have not provided an effective means for limiting the amount of bone material removed.

More specifically, with respect to adjacent vertebral bones, it is highly desirable to remove the thin CFC layer while leaving a maximum thickness of the cortical bone endplate intact for secure and stable remodeling ingrowth fixation or fusion with an implanted spacer element. By contrast, if the cortical bone endplate is removed entirely or substantially by a bone removal device, softer and less-stable cancellous bone is exposed for ingrowth attachment with the spacer element to result in a much weaker bone-spacer element fusion strength.

There exists, therefore, a significant need for further improvement in and to devices for removing bone and bone-like material, such as a thin CFC layer from adjacent intervertebral bone structures, in a manner which closely monitors and thereby prevents removal of excess bony material. The present invention fulfills these needs, and provides further related advantages.

### SUMMARY OF THE INVENTION

In accordance with the invention, an intervertebral milling instrument is provided for closely controlled removal of a thin layer of calcified fibrous cartilage (CFC) from adjacent vertebrae during spinal fusion or spacer element implantation surgery. The milling instrument comprises a guide housing having a closely predetermined thickness dimension sized to fit into the specific and unique patient intervertebral space following surgical removal of natural cartilage therefrom. A grinding wheel is rotatably carried within the guide housing in a manner for closely controlled and limited exposure of the grinding wheel at opposite side faces thereof for controlled and limited removal of thin CFC layers from adjacent cortical end plates lining the intervertebral space. In a preferred form, the limited exposure of the grinding wheel is effective to remove about 100 to about 250 microns of bony material from the adjacent vertebrae, thereby substantially removing the thin CFC layers without excessive removal of cortical endplate bone. Accordingly, the prepared cortical end plate bones lining the intervertebral space effectively remodel post-surgically for strong ingrowth fusion attachment to an implanted spacer element.

The grinding wheel is rotatably driven as by attachment to a surgical drill, preferably to include supply of a saline solution to and suction removal from the grinding sites. The saline solution prevents undesirable overheating at the milling or grinding site to prevent bone necrosis, whereas the suction source beneficially removes the saline solution and milled-off CFC particulate from the patient. In one preferred form, the grinding wheel is oriented in-line for rotatable driving by a conventional surgical drill. In an alternative preferred form, the grinding wheel is rotatably driving on an axis that is perpendicular to a rotary drill axis. In both embodiments, the guide housing limits grinding wheel exposure at opposite sides of the guide housing for limited engagement with and removal of adjacent bony structures such as the thin CFC layers.

In a preferred form, the improved milling instrument of the present invention is provided in kit form, with several different guide housing thicknesses suitable for use with different intervertebral spacing unique to each patient and the location of the surgical site along the patient's spine. That is, in addition to dimensional variations unique to each patient, the invention recognizes that dimension of the intervertebral space as well as the average thickness of the associated CFC layers varies according to the cervical, thoracic, or lumbar location of the surgical site. By providing multiple different-thickness guide housings and different associated grinding wheel exposure distances, a surgeon can selected a suitably sized milling instrument dimensioned for effective removal of the CFC layers preparatory to implantation of a selected spacer element.

Other features and advantages of the present invention will become apparent from the following more detailed description, taken in connection with the accompanying drawing which illustrate, by way of example, the principals of the present invention.

### BRIEF DESCRIPTION OF THE DRAWINGS

The accompanying drawings illustrate the invention. In such drawings:

FIG. 1 is a perspective view, shown partially in schematic form, illustrating a intervertebral milling instrument constructed in accordance with a preferred form of the invention;

FIG. 2 is an enlarged sectional view taken generally on the line 2-2 of FIG. 1;

FIG. 3 is a perspective view similar to FIG. 1, but showing the intervertebral milling instrument with a portion of a guide housing removed to reveal internal construction details thereof;

FIG. 4 is an electron microscope image depicting adjacent patient vertebrae defining an intervertebral space therebetween;

FIG. 5 is an enlarged electron microscope image depicting a portion of FIG. 4, and showing a calcified fibrous cartilage (CFC) layer overlying a cortical endplate at one side of the intervertebral space;

FIG. 6 is a further enlarged electron microscope image depicting a portion of FIG. 5, and illustrating the CFC layer in more detail;

FIG. 7 is a graphic depiction of empirical test data showing the average thickness of a CFC layer formed on a cortical endplate lining an intervertebral space, in accordance with the cervical, thoracic or lumbar location of the intervertebral space along a patient's spine;

FIG. 8 is a somewhat schematic representation of a portion of a spinal lumbar region including a degenerating or diseased cartilage-based spinal disk;

FIG. 9 is a somewhat schematic representation similar to FIG. 8, but illustrating an exemplary spacer element implanted into the intervertebral space following removal of the cartilage-based spinal disk, and further showing supplemental pedicle screw fixation means;

FIG. 10 is a somewhat schematic representation depicting use of the milling instrument of the present invention to grind or shave off the CFC layers formed on the cortical endplates of adjacent vertebrae lining the intervertebral space;

FIG. 11 is a schematic view showing a kit comprising multiple milling instruments of the present invention with different thickness dimensions for selection of one milling instrument in accordance with unique patient dimensions;

FIG. 12 is a perspective view similar to FIG. 1, but showing the milling instrument in accordance with one alternative preferred form of the invention; and

FIG. 13 is a perspective view similar to FIG. 3, but illustrating the alternative embodiment of FIG. 12 with a portion of a guide housing removed to reveal internal construction details thereof.

### DETAILED DESCRIPTION OF THE PREFERRED EMBODIMENTS

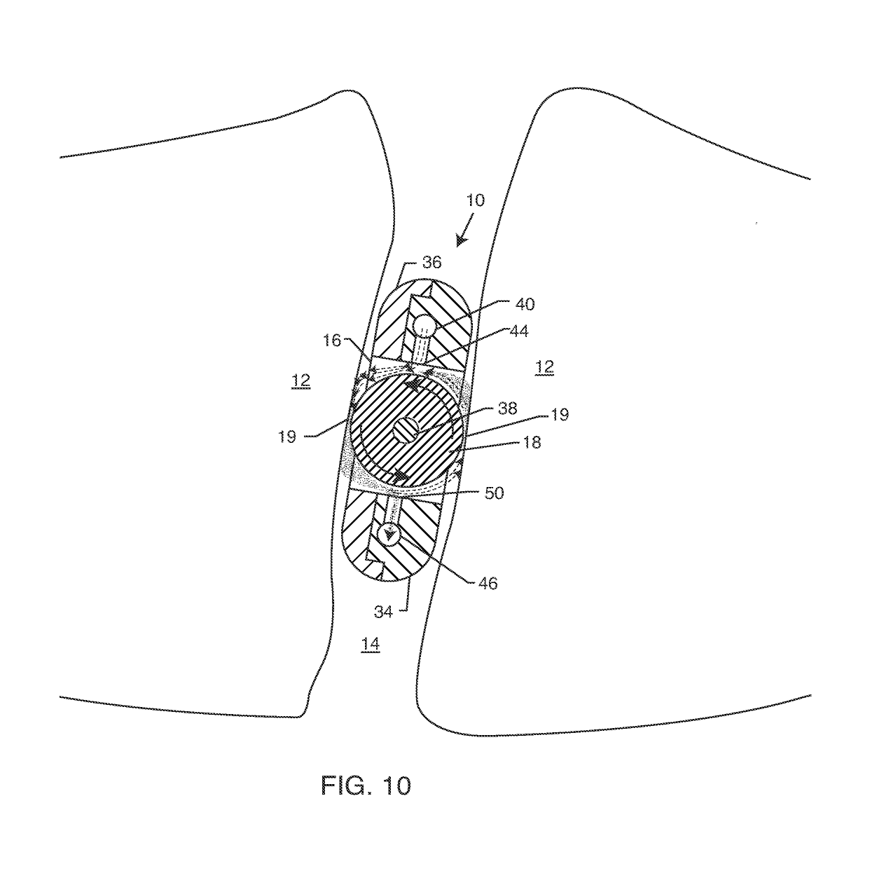

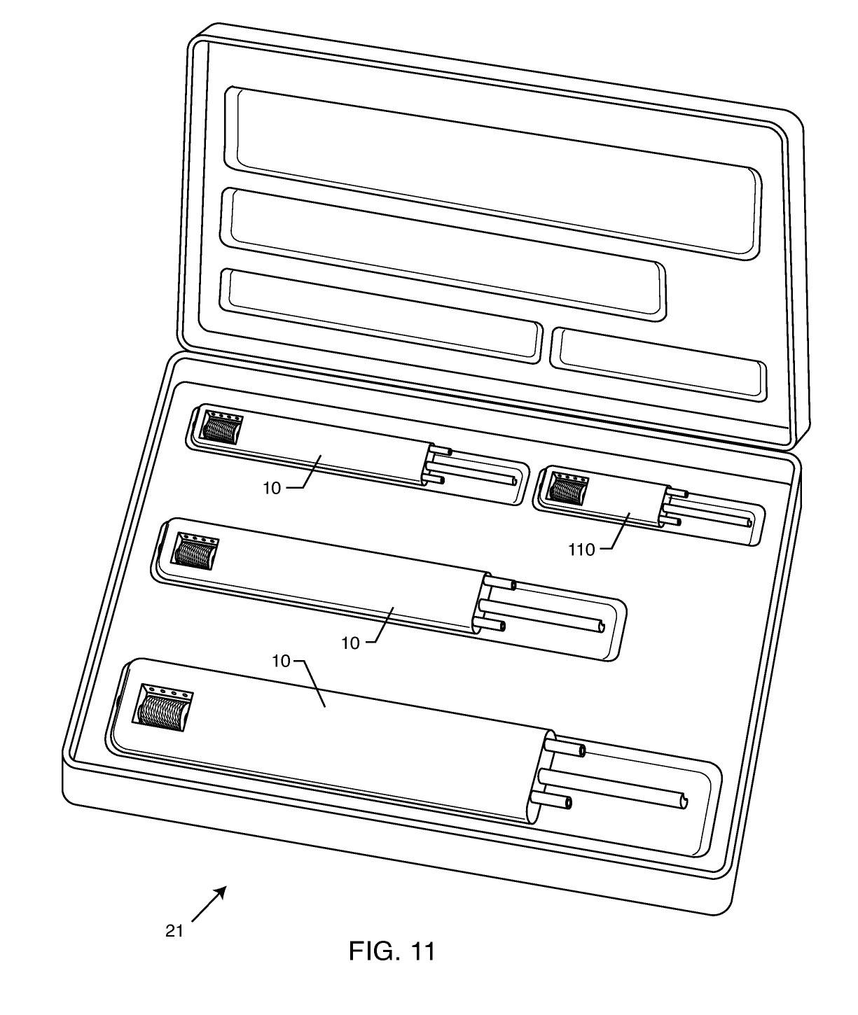

As shown in the exemplary drawings, a milling instrument referred to generally by the reference numeral 10 is provided for grinding or shaving in a closely controlled manner a thin layer from the cortical bone endplates 12 (FIG. 10) lining an intervertebral space 14 in the course of spinal fusion surgery or the like. The milling instrument 10 comprises a guide housing 16 having a thickness dimension selected to fit with close tolerance into the intervertebral space, in combination with a grinding or milling wheel 18 exposed and protruding a short distance beyond the opposed side faces of the guide housing 16 for engaging and contacting the adjacent cortical endplates 12 lining the intervertebral space 14. The grinding wheel 18 thereby removes a thin layer such as a thin layer of calcified fibrous cartilage 19 (CFC) from the cortical endplates 12 in a controlled manner, for achieving improved fusion ingrowth fixation with a spacer element 20 (FIG. 9) implanted subsequently into the intervertebral space 14. In a preferred form, the milling instrument 10 is provided in a kit 21 (FIG. 11) including multiple milling instruments 10 of different thickness dimensions and different grinding wheel exposure distances, so that a surgeon can select a specific milling instrument 10 having dimensions suitable for use on a unique patient.

Spinal surgery has in recent years become popular for relieving patient back pain associated with degenerative spinal disk disease or spinal disk injury, particularly in the lower or lumbar region of the spine. In this regard, spinal surgery typically identifies and removes a cartilage-based disk 22 (FIG. 8) which has become herniated and/or partially collapsed to result in nerve compression and associated patient back pain. The spinal surgery seeks to access and remove the diseased cartilage-based disk 22, and to install a spacer element 20 (FIG. 9) into the resultant intervertebral space 14. Supplemental fixation devices comprising bone plates 24 and pedicle screws 26 are often employed in combination with the spacer element 20.

Fusion or bone ingrowth attachment between the spacer element 20 and the cortical endplates 12 of the adjacent vertebrae bones 28 and 30 lining the intervertebral space 14 is normally required for long-term implant stability without complications, such as undesired migration of the spacer element 20 toward the patient's spinal cord, or in an opposite direction for movement out of the intervertebral space. While FIG. 9 shows one exemplary type of spacer element 20, persons skilled in the art will recognize and appreciate that other types of spacer elements can be used, including but not limited to, mobile bearing devices and/or U-shaped spacer elements with an interior region packed with autologous or autogenous bone chips, and the like. In each case, secure and stable fusion attachment of between the spacer element and the adjacent bone structures is highly desirable.

The present invention recognizes that the adjacent bones 28 and 30 lining the intervertebral space 14 carry a relatively thin coating or layer 19 comprising calcified fibrous cartilage (CFC) material that is highly beneficial with respect to securely attaching the cartilage-based disk 22 to the bones 28, 30. Upon removal of a diseased disk 22 from the intervertebral space 14, this thin layer or coating 19 of CFC material remains on the cortical endplates 12 of the adjacent vertebral bones 28 and 30. It has been recognized that these thin layers of CFC material tend to resist bone remodeling and healing, and resultant bone ingrowth attachment to a subsequently implanted spacer element 20. That is, while the CFC material is beneficial to attaching the cartilage-based disk 22 to the bones 28, 30, it is detrimental to secure and stable ingrowth attachment of an implanted spacer element 20 to the bones 28, 30.

The present invention provides the milling instrument 10 for grinding or shaving off of the thin CFC layers 19 from the cortical endplates 12 of the adjacent bones 28, 30 lining the intervertebral space 14, for improved bone ingrowth fusion attachment with a subsequently implanted spacer element 20. Importantly, the milling instrument 10 is designed and functions to remove a closely controlled thin layer from the cortical endplate 12 of each adjacent bone 28 or 30, thereby effectively removing the CFC layer 19 without excessive removal of hard cortical bone from the endplate 12. Specifically, the thickness dimension of the guide housing 16 is closely selected to fit with a close tolerance into the intervertebral space 14, and the grinding wheel 18 is exposed and protrudes slightly beyond the opposite side faces of the guide housing 16 for extremely limited and closely controlled removal of the bone-like CFC layers 19 without excessive removal of cortical bone. In a preferred form, the grinding wheel exposure is limited to about 150 microns on each side of the guide housing 16 for shaving the adjacent cortical endplates 12 by up to about 100 to about 250 microns, and more preferably about 150 microns, in the lumbar region of the patient's spine. By contrast, excessive removal of the cortical endplate bone is specifically avoided, wherein such excessive removal can otherwise weaken the resultant ingrowth attachment strength as by exposing softer cancellous bone.

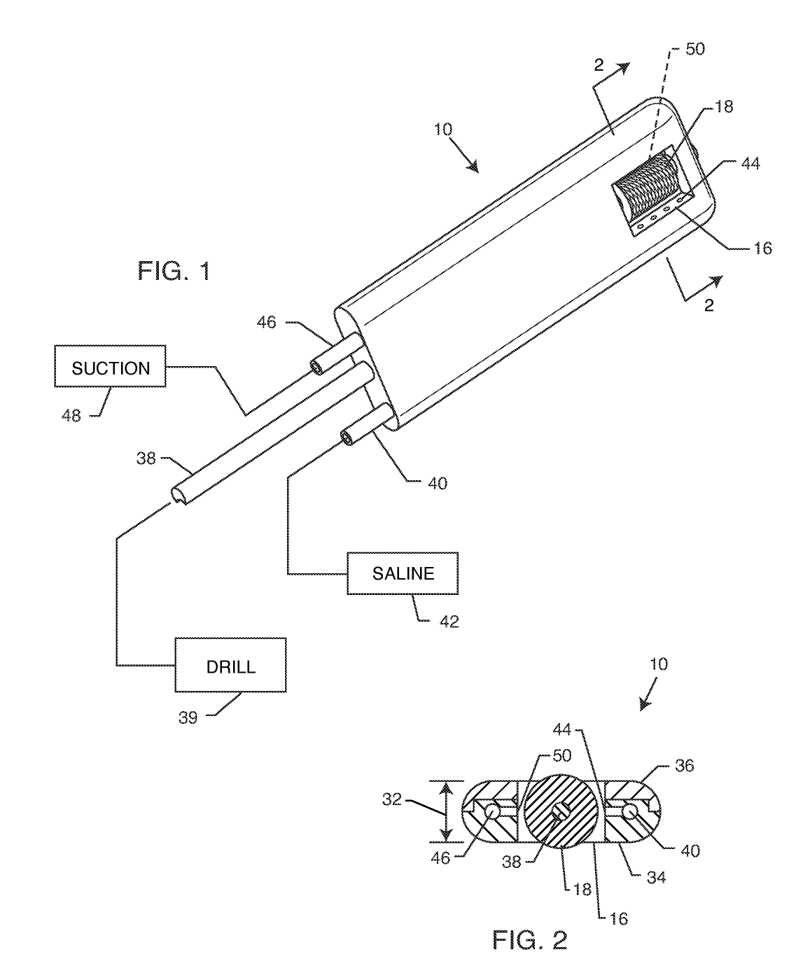

FIGS. 1-3 show the improved milling instrument 10 in one preferred form, to comprise the guide housing 16 rotatably supporting the grinding wheel 18. As shown, the guide housing 16 has a carefully controlled and carefully selected thickness dimension (referenced by arrow 32) chosen to fit with a close tolerance into the intervertebral space 14 (FIG. 10). This guide housing 16 is, in the preferred form, constructed from base and top housing members 34 and 36 of molded plastic or the like and adapted for assembly as by snap-fitting or the like (FIG. 2). A main rotary drive shaft 38 extends longitudinally through the base housing member 34 and is coupled to the grinding wheel 18 for direct rotary driving thereof. A proximal end of the rotary shaft 38 protrudes from an aft or proximal end of the guide housing 16 for suitable attachment to and rotary driving by a standard or conventional surgical drill 39 (FIG. 1). Accordingly, the surgical drill rotatably drives the shaft 38, which in turn rotatably drives the grinding wheel 18. FIGS. 1-3 show a preferred embodiment of the invention with the grinding wheel 18 driven directly or coaxially with the rotary shaft 38.

The grinding wheel 18 has a relatively round outside diameter surface for grinding or milling the thin CFC layers from the cortical endplates 12 of the adjacent patient bones 28, 30. As shown best in FIG. 2, the grinding wheel 18 is exposed and protrudes slightly a short distance beyond the opposed side faces of the guide housing 16 for controlled depth removal of the thin CFC layers 19 from the adjacent bones 28, 30 lining the intervertebral space 14.

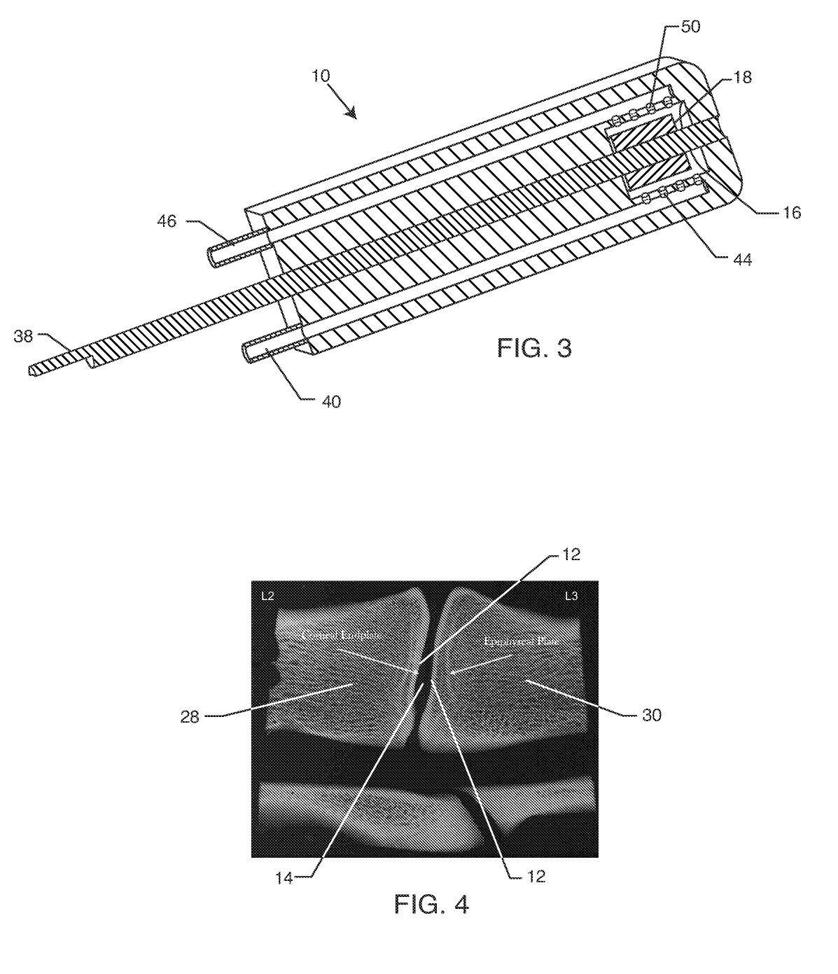

A saline tube 40 is also supported by the base housing member 34 and includes an upstream or proximal end exposed for suitable attachment to a saline fluid supply source 42 (FIG. 1). This saline fluid supply is pumped through the saline tube 40 to a distal end coupled within the guide housing to a multi-ported saline manifold 44 (FIG. 3) disposed at one side of the grinding wheel 18 for fluidizing the bone-like CFC layers 19 removed or ground from the adjacent bones 28, 30, and also to cool the grinding site to prevent undesired necrosis of the bony tissue.

A suction tube 46 is supported by the base housing member 34 and also defines a downstream or proximal end exposed for suitable attachment to a suction source 48 (FIG. 1). In use, this suction source 48 draws the saline solution and fluidized grinding particulate from the grinding site through a multi-ported suction manifold 50 (FIG. 3) disposed adjacent to the grinding wheel 18 at a position opposite to the saline manifold 44, and coupled to a distal end of the suction tube 46 within said guide housing. Thus, the saline solution and fluidized grinding particulate are suction-drawn further through the suction tube 46 for collection and disposal at the suction source 48.

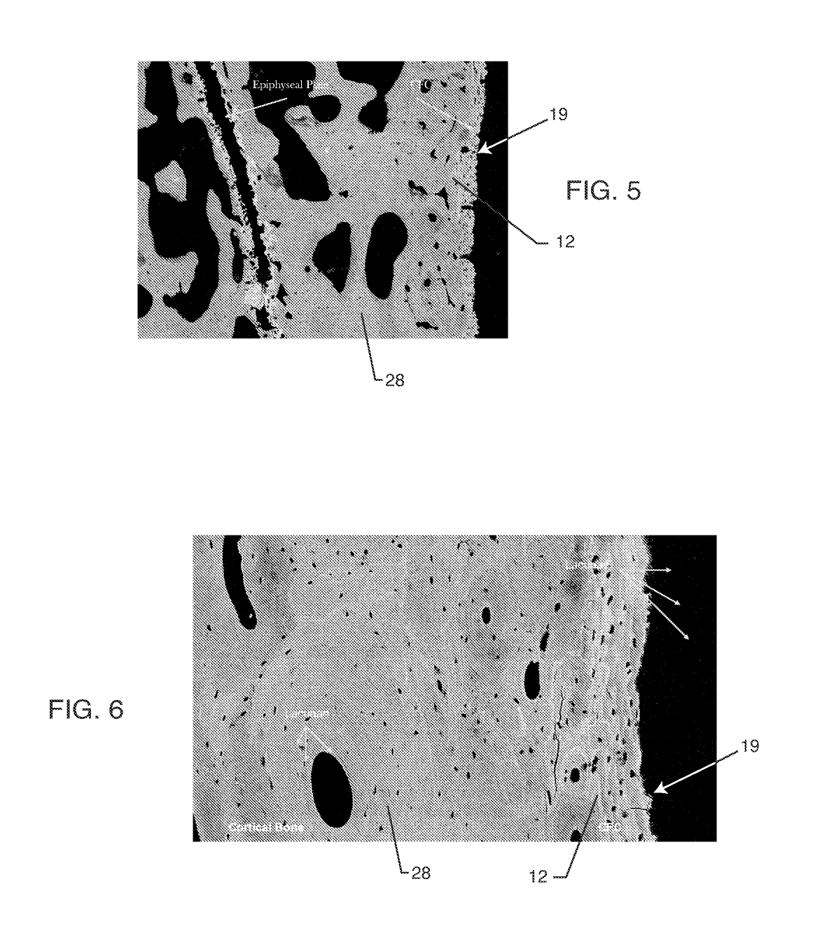

FIGS. 4-6 comprise electron microscope images of sheep bones chosen for their similarity to human spinal vertebrae. FIG. 4 shows the adjacent spinal vertebral bones 28 and 30 in normally slightly spaced-apart relation by virtue of a natural cartilage-based disk 22 (FIG. 8). FIG. 4 also shows the cortical endplates 12 of the opposed bones 28, 30 lining the intervertebral space 14. FIGS. 5 and 6 are progressively enlarged electron microscope images of the vertebral bone 28 showing a thin layer 19 of calcified fibrous cartilage (CFC) in relatively small and hard platelet-like form coating the cortical endplate 12 of the bone 28.

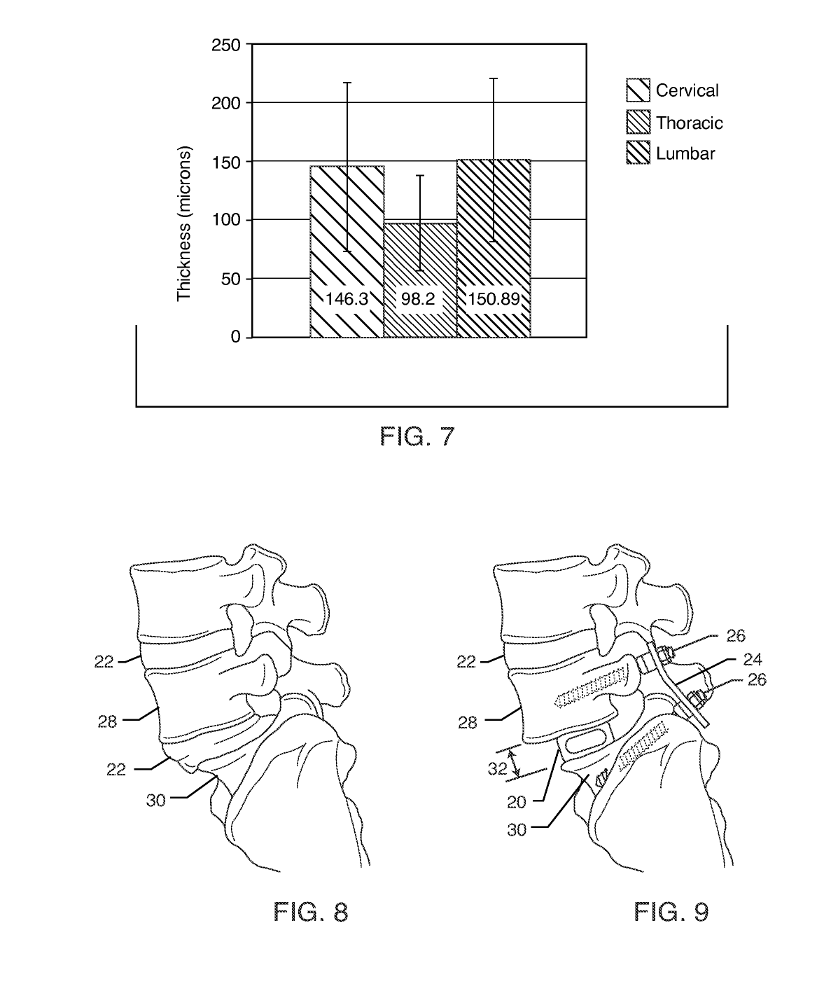

FIG. 7 is a graphical representation indicating an empirically determined average thickness of the CFC coating 19 (in microns) on the vertebral bones of a sheep spine, in accordance with the cervical (upper), thoracic (middle), or lumbar (lower) region of those vertebrae along the patient's spine. As evidenced by this empirical chart, the average thickness of the CFC coating 19 in the cervical (upper) and lumbar (lower) regions is about 150 microns. By contrast, the average thickness of the CFC coating 19 in the thoracic (middle) region of the patient's spine is about 100 microns. It is noted that the majority of spinal fusion surgeries occur in the lumbar (lower) region, with the cervical (upper) region constituting the next most common region for spinal fusion surgeries.

FIGS. 8 and 9 illustrate a lumbar region of the human spine with a diseased or injured cartilage-based disk 22 (FIG. 8) and an exemplary prosthesis 52 (FIG. 9) having a spacer element 20 implanted into the intervertebral space 14 following removal of the diseased disk 22.

FIG. 10 shows use of the milling instrument 10 of the present invention to grind or shave off the thin CFC layers 19 from the cortical endplates 12 of the adjacent overlying and underlying vertebral bones 28, 30 lining the intervertebral space 14, following removal of a diseased or injured cartilage-based disk 20. As shown, the guide housing 16 has a carefully selected thickness dimension chosen for close tolerance reception into the intervertebral space 14 between the adjacent vertebral bones 28, 30. In this position, the grinding wheel 18 is rotatably driven to engage and shave off the thin and hard CFC layers 19, without excessive grinding into or removal of the bony structures of the cortical endplates 12. In the case of a lumbar (lower) or cervical (upper) region of the patient's spine, the grinding wheel 18 extends or protrudes outwardly beyond the opposite side faces of the guide housing by a limited distance of about 100 to about 250 microns, and more preferably about 150 microns, to accommodate substantial removal of the CFC layers 19, but without excess remove of the bony tissue comprising the cortical endplates 12. The saline and suction tubes 40 and 46 are suitably coupled to the respective saline and suction sources 42 and 48 for effective cooling of the grinding sites and for effective removal of grinding particulate from the patient.

FIG. 11 shows a preferred kit 21 comprising a plurality of milling instruments 10 each having a guide housing formed with a different physical thickness dimension and/or a different degree or distance of grinding wheel exposure at opposite sides of the guide housing. With such kit 21, a surgeon may select a milling instrument 10 having a particular thickness dimension suitable to fit with close tolerance into the intervertebral space 14 defined by a specific patient anatomy, in combination with a particular grinding wheel exposure distance at opposite sides of the guide housing for shaving off the CFC layers to a selected and controlled depth according to the specific top-to-bottom location of the surgical site along the patient's spine.

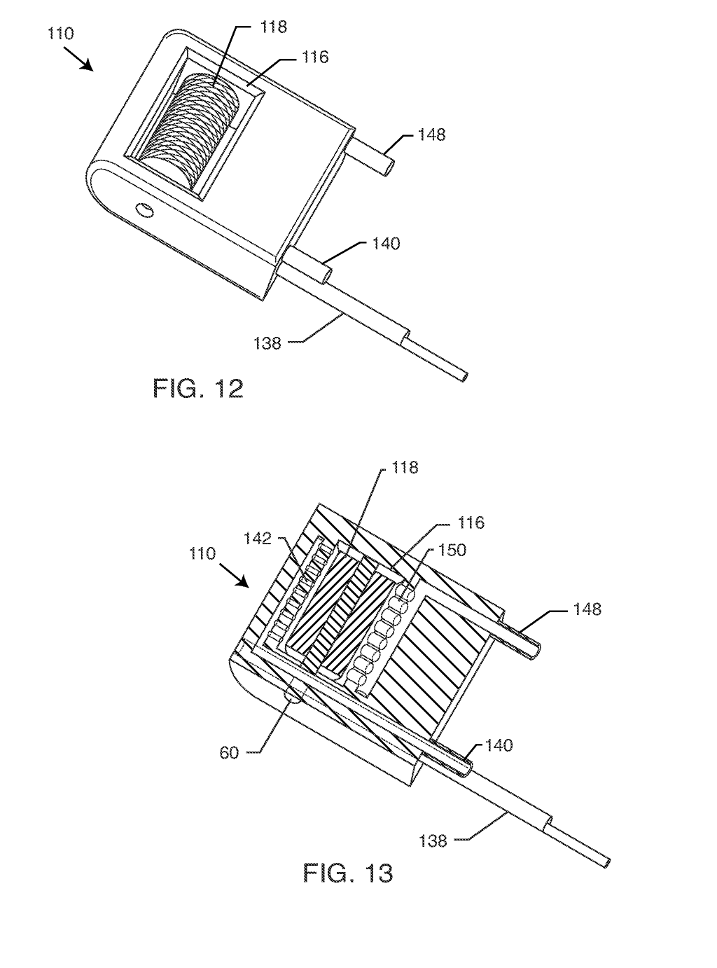

FIGS. 12 and 13 show an alternative preferred embodiment of the milling instrument, wherein components similar to those shown and described with respect to the embodiment depicted in FIGS. 1-3 are identified by common reference numerals increased by 100. As shown, the modified milling instrument 110 comprises a guide housing 116 of selected thickness dimension for close tolerance insertion to the intervertebral space 14, in combination with a grinding wheel 118 exposed a short distance at opposite sides of the guide housing 116. In FIGS. 12-13, the grinding wheel 118 is oriented substantially perpendicular to an axis of an elongated rotary drive shaft 138. Specifically, as shown, the rotary shaft 138 is carried near one side edge of the guide housing 116 and protrudes from a proximal end of the guide housing 116 for suitable rotary driven attachment to a standard surgical drill (not shown) or the like. A transmission element 60 such as a worm gear 60 or the like is mounted within the guide housing 116 at one end of the grinding wheel 118 to rotatably driving the grinding wheel 118 in response to rotation of the drive shaft 138, as previously described.

FIGS. 12 and 13 also show a pair of saline supply and suctions tubes 140 and 146 for communicating with the grinding sites by means of ported manifolds 144 and 150, respectively. Persons skilled in the art will appreciate that this modified milling instrument 110 may be provided in the form of a kit (not shown) comprising multiple different-sized instruments 110 is the same manner as the kit 21 shown in FIG. 11. Alternately, if desired, multiple different-sized milling instruments 110 may form part of the kit 21 shown in FIG. 11.

Although various embodiments and alternatives have been described in detail for purposes of illustration, various further modifications may be made without departing from the scope and spirit of the invention. Accordingly, no limitation on the invention is intended by way of the foregoing description and accompanying drawings, except as set forth in the appended claims.

What is claimed is:

1. An intervertebral milling instrument, comprising:
a guide housing having a selected thickness dimension for close tolerance reception into an intervertebral space between adjacent vertebral bones following surgical removal of a cartilage-based disk from said intervertebral space;
a grinding wheel carried by said guide housing, said grinding wheel being exposed and protruding slightly beyond opposite side faces of said guide housing; and
means for rotatably driving said grinding wheel when said guide housing is positioned within said intervertebral space for controlled removal of a thin layer from the adjacent vertebral bones.

2. The intervertebral milling instrument of claim 1, wherein said grinding wheel protrudes beyond each of said opposite side faces of said guide housing by a distance of about 100 to about 250 microns.

3. The intervertebral milling instrument of claim 2, wherein said grinding wheel protrudes beyond each of said opposite faces of said guide housing by a distance of about 150 microns.

4. The intervertebral milling instrument of claim 1, wherein said grinding wheel protrudes beyond each of said opposite side faces of said guide housing by a distance selected for removal of a thin calcified fibrous cartilage (CFC) layer from the adjacent vertebral bones, substantially without excess removal of cortical endplates from said adjacent vertebral bones.

5. The intervertebral milling instrument of claim 1, wherein said means for rotatably driving said grinding wheel comprises a rotary drive shaft carried by said guide housing.

6. The intervertebral milling instrument of claim 5 wherein said grinding wheel is supported by said guide housing for rotation on an axis substantially coaxial with a rotary axis of said drive shaft.

7. The intervertebral milling instrument of claim 5 wherein said grinding wheel is supported by said guide housing for rotation on an axis substantially perpendicular to a rotary axis of said drive shaft, and further including a transmission element coupled between said drive shaft and said grinding wheel for rotatably driving said grinding wheel in response to drive shaft rotation.

8. The intervertebral milling instrument of claim 1 further comprising a saline tube carried by said guide housing, said saline tube having a proximal end for suitable connection to a saline fluid source, and a distal end coupled within said guide housing to a ported saline manifold disposed adjacent to said grinding wheel.

9. The intervertebral milling instrument of claim 8 further comprising a suction tube carried by said guide housing, said suction tube having a proximal end for suitable connection to a suction source, and a distal end coupled within said guide housing to a ported suction manifold disposed adjacent to said grinding wheel, said ported saline and said ported suction manifolds being disposed generally at opposite sides of said grinding wheel.

10. An intervertebral milling instrument, comprising:
a guide housing having a selected thickness dimension for close tolerance reception into an intervertebral space between adjacent vertebral bones following surgical removal of a cartilage-based disk from said intervertebral space;
a grinding wheel carried by said guide housing, said grinding wheel being exposed and protruding slightly beyond opposite side faces of said guide housing;
a saline tube carried by said guide housing, said saline tube having a proximal end for suitable connection to a saline fluid source, and a distal end coupled within said guide housing to a ported saline manifold disposed adjacent to said grinding wheel;
a suction tube carried by said guide housing, said suction tube having a proximal end for suitable connection to a suction source, and a distal end coupled within said guide housing to a ported suction manifold disposed adjacent to said grinding wheel, said ported saline and said ported suction manifolds being disposed generally at opposite sides of said grinding wheel; and
means for rotatably driving said grinding wheel when said guide housing is positioned within said intervertebral space for controlled removal of a thin layer from the adjacent vertebral bones;
said grinding wheel protruding beyond each of said opposite side faces of said guide housing by a distance selected for removal of thin calcified fibrous cartilage (CFC) layers from the adjacent vertebral bones, substantially without excess removal of cortical endplates from said adjacent vertebral bones.

11. The intervertebral milling instrument of claim 10, wherein said grinding wheel protrudes beyond each of said opposite side faces of said guide housing by a distance of about 100 to about 250 microns.

12. The intervertebral milling instrument of claim 11, wherein said grinding wheel protrudes beyond each of said opposite side faces of said guide housing by a distance of about 150 microns.

13. The intervertebral milling instrument of claim 10, wherein said means for rotatably driving said grinding wheel comprises a rotary drive shaft carried by said guide housing.

14. The intervertebral milling instrument of claim 13 wherein said grinding wheel is supported by said guide housing for rotation on an axis substantially coaxial with a rotary axis of said drive shaft.

15. The intervertebral milling instrument of claim 13 wherein said grinding wheel is supported by said guide housing for rotation on an axis substantially perpendicular to a rotary axis of said drive shaft, and further including a transmission element coupled between said drive shaft and said grinding wheel for rotatably driving said grinding wheel in response to drive shaft rotation.

16. A milling instrument kit, comprising:
a plurality of milling instruments each having a guide housing with a selected different thickness dimension for reception into an intervertebral space between adjacent vertebral bones following surgical removal of a cartilage-based disk from said intervertebral space of a specific surgical patient, a grinding wheel carried by said guide housing and being exposed and protruding slightly beyond opposite side faces of said guide housing, and means for rotatably driving said grinding wheel when said guide housing is positioned within said intervertebral space for controlled removal of a thin layer from the adjacent vertebral bones;
one of said plurality of milling instruments having a thickness dimension for close tolerance reception into said intervertebral space, and having said grinding wheel protruding slightly beyond said opposite side faces of said guide housing by a dimension suitable for removal of thin calcified fibrous cartilage (CFC) layers from the adjacent vertebral bones, substantially without excess removal of cortical endplate bone from said adjacent vertebral bones.

17. The milling instrument kit of claim 16, wherein said grinding wheel of said plurality of milling instruments protrudes beyond each of said opposite side faces of said guide housing by different distances of about 100 to about 250 microns.

18. The intervertebral milling instrument of claim 17, wherein said grinding wheel protrudes beyond each of said opposite side faces of said guide housing by a distance of about 150 microns.
